SEQUENCE LISTING (2 sequences)

SEQ ID NO: 1 - RNA, 33 nt, synthetic construct
  misc_feature 1..33: note = MMLV-RT RNA aptamer
  source 1..33: mol_type = other RNA
ttaccacgcgctcttaactgctagcgccatggc

SEQ ID NO: 2 - DNA, 41 bp, synthetic construct
  misc_feature 1..41: note = MMLV-RT aptamer inhibitor
  source 1..41: mol_type = other DNA
agttttggccatggcgctagcagttaagagcgcgtggtaag

(19) United States
(12) Patent Application Publication
(10) Pub. No.: US 2025/0051836 A1
(43) Pub. Date: Feb. 13, 2025

(54) METHOD FOR AMPLIFYING RNA IN A SAMPLE

(71) Applicant: QIAGEN GmbH, Hilden (DE)

(72) Inventor: Colin DONOHOE, Köln (DE)

(21) Appl. No.: 18/721,259

(22) PCT Filed: Nov. 25, 2022
(86) PCT No.: PCT/EP2022/083339

§ 371 Date: Jun. 18, 2024

(30) Foreign Application Priority Data
Dec. 21, 2021 (EP) 21216371.1

(51) Int. Cl. C12Q 1/6848

(52) U.S. Cl. CPC C12Q 1/6848

(57) ABSTRACT

The present invention relates to a method for amplifying a target RNA in a sample, an oligonucleotide usable in the method according to the invention, a kit for amplifying a target RNA, and a use of an oligonucleotide for inhibiting an aptamer oligonucleotide.

The present invention relates to a method for amplifying a target RNA in a sample, an oligonucleotide usable in the method according to the invention, a kit for amplifying a target RNA, and a use of an oligonucleotide for inhibiting an aptamer oligonucleotide.

### FIELD OF THE INVENTION

The present invention relates to the field of molecular biology, more particularly to the amplification of nucleic acid molecules, and, specifically, to the amplification of target RNA in a sample by reverse transcription (RT) and polymerase chain reaction (PCR).

### BACKGROUND OF THE INVENTION

Reverse transcription polymerase chain reaction (RT-PCR) is a laboratory technique combining reverse transcription of RNA into DNA, in this context called complementary DNA or cDNA, and amplification of specific DNA targets using polymerase chain reaction (PCR). RT-PCR is perhaps the most widespread method for the quantification and/or detection of RNA targets in the repertoire of molecular biology. In the first step of RT-PCR, termed reverse transcription (RT), a reverse transcriptase enzyme, e.g. Moloney murine leukemia virus reverse transcriptase (MMLV-RT), a mixture of deoxyribonucleoside triphosphates (dNTPs), and oligonucleotide primers are used to convert an RNA template into complementary DNA (cDNA). To promote specific oligonucleotide priming, high RT temperatures, e.g. 50° C., are ideally used for the RT incubation step. Subsequently, a thermostable DNA-dependent DNA polymerase, a mixture of deoxyribonucleoside triphosphates (dNTPs) and oligonucleotide primers are used to amplify and detect the cDNA in a polymerase chain reaction (PCR).

One-step RT-PCR is a version of RT-PCR in which the two reactions that comprise RT-PCR (reverse transcription and PCR) are performed within a single reaction mixture using a single thermocycling protocol. By combining the RT and PCR reactions into one reaction mix, the one-step RT-PCR method shortens the time to result, makes the RT-PCR workflow more convenient, and limits the risk of human error by eliminating sample handling steps.

One-step real-time RT-PCR (one-step RT-qPCR) and one-step digital RT-PCR (one-step RT-dPCR) are versions of one-step RT-PCR in which target specific probes (oligonucleotides) labelled with fluorophores or intercalating dyes are used to detect the amplification of cDNA in real-time (one-step RT-qPCR) or within discrete reaction partitions at end of cycling (one-step RT-dPCR).

RT-PCR, one-step RT-qPCR and one-step RT-dPCR are regarded as methods with high sensitivity and specificity. Nonetheless, specificity and sensitivity of these methods are challenged, in part, by two factors during the reverse transcription step: the relatively high activity of reverse transcriptase enzymes at ambient temperatures (i.e. 22° C.), and the non-specific binding of oligonucleotide primers that can occur at these ambient temperatures. Combined, these two factors can result in cDNA products being generated by the elongation of misprimed primers, which competes with the synthesis of specific cDNA. Ultimately, these non-specific products can decrease sensitivity as well as specificity of RT-PCR assays.

Owing to these concerns, guidelines for one-step RT-PCR experiments call for the assembly of reaction mixtures at cold temperatures, i.e. on ice, and for immediate RT-PCR cycling once reaction mixtures are fully assembled. Both of these constraints are inconvenient to one-step RT-PCR users, even those with low throughput, i.e. single plate workflows. Furthermore, these guidelines are no guarantee that the issues of specificity and sensitivity will not affect a one-step RT-PCR experiment. RT enzymes may be active in reaction mixtures even when assembled on ice. Also, the time needed to transfer the one-step RT-PCR reaction mixtures into a thermocycler may be enough time for the RT enzyme to reverse transcribe misprimed RNA.

Furthermore, RT mispriming at ambient temperature is of particular concern in workflows that result in a time delay between one-step RT-PCR reaction mixture assembly and thermocycling. This can occur in workflows where reaction mixture assembly is performed in high throughput but thermocycler capacity and/or throughput is a limiting bottleneck. Accordingly, such workflows are either avoided altogether or performed with the risk of specificity and sensitivity issues described above.

The currently used RT digital PCR (dPCR) instrument portfolio, such as QIAcuity®, QIAcuity Four and QIAcutiy Eight (Qiagen, Hilden, Germany), allows users to simultaneously load four and eight dPCR reaction plates into a dPCR device, respectively. Users can also simultaneously start the cycling programs for all of the loaded plates. However, these RT-PCR devices often have only one or two thermocycler units, respectively. Thus, while the loaded plates are all “started” in parallel, the plates are in fact cycled successively. As a result, there can be a significant time delay between one-step RT-PCR reaction mix assembly and the thermocycling of one-step RT-dPCR plates in a multiplate workflow. With current RT-dPCR cycling programs, approximately 6 hours pass between master mix assembly and thermocycling of the last plate in four-plate and eight-plate workflows. However, even a single plate requires approximately 20 min of priming and rolling before proceeding into RT-dPCR cycling.

The temperatures inside the common RT-dPCR instrument have been determined to be 12 to 14° C. above ambient temperatures. Assuming a typical lab temperature of 23° C., the last dPCR reaction plate in a four-plate or eight-plate multiplate workflow waits in the RT-dPCR instrument for about 6 hours at approx. 37° C. before thermocycling begins. RT enzymes are highly active at temperatures around 37° C., but these temperatures are not high enough to ensure specific priming of oligonucleotides. Therefore, multiplate workflows are at great risk of misprimed RNA reducing specificity and sensitivity.

Given the challenges and concerns that accompany RT-PCR and one-step RT-PCR methods, technical solutions that universally minimize or eliminate transcription of misprimed RNA prior to RT-PCR cycling have been highly desirable. One possible technical solution to the problem would target the activity of the RT enzyme, such that the RT enzyme is inactive or minimally active at lower temperatures but fully active at desired RT temperatures (e.g. 50° C.).

One potential source of universal and reversible inhibition of RT activity are aptamers. Aptamers are oligonucleotide or peptide molecules that bind to a specific target molecule. Single-stranded oligonucleotide aptamers assume a variety of shapes due to their tendency to form helices and single-stranded loops. They bind to their targets with high selectivity and specificity. Aptamers with affinity for a desired target are selected from a large oligonucleotide library through a process called SELEX, which stands for ‘Sequential Evolution of Ligands by Exponential Enrichment’. New England Biolabs® offers a “warm-start” one-step RT-qPCR mastermix that uses heat-sensitive, reversibly bound DNA aptamers licensed from Somalogic.

The New England Biolabs® aptamer solution uses DNA aptamers to reduce RT function to almost zero at temperatures below 40° C. while allowing full RT function at temperatures higher temperatures, i.e. 50° C.

Such aptamer-based approaches are also known from Chen and Gold (1994), Selection of high-affinity RNA ligands to reverse transcriptase: inhibition of cDNA synthesis and RNase H activity, Biochemistry 33, pp. 8746-8756; Rutschke et al. (2015), Hot start reverse transcriptase: an approach for improved real-time RT-PCR performance, Journal of Analytical Science and Technology 6, pp. 1-5.

RT-PCR approaches trying to address the problems stated above with or without aptamers are also known from WO 2013/024064 and WO 2017/079636.

The inventors have realized that the aptamers used in the known RT-PCR approaches also inhibit RT activity at optimal and desired temperatures (about 50° C.) when compared to control reactions without aptamer, resulting in an unsatisfactory performance of the RT-PCR.

Against this background it is an object underlying the invention to provide an improved RT-PCR where the disadvantages of the prior methods are avoided or at least reduced. In particular such an RT-PCR is to be provided with increased sensitivity and specificity as compared to current methods in the art.

The present invention satisfies these and other needs.

### SUMMARY OF THE INVENTION

The present invention provides a method for amplifying a target RNA in a sample comprising the following steps:
(i) providing a reaction mixture at a first temperature, said reaction mixture comprising:
   the sample;
   a reverse transcriptase enzyme;
   a DNA polymerase enzyme;
   a mixture of deoxyribonucleoside triphosphates (dNTPs);
   at least one primer configured to convert the target RNA into complementary DNA (cDNA) and/or to amplify said cDNA;
   an aptamer oligonucleotide configured to specifically inhibit the reverse transcriptase enzyme, and
   an inhibitor of the aptamer oligonucleotide,
(ii) reverse transcribing said target RNA into cDNA by incubating said reaction mixture at a second temperature higher than said first temperature resulting in an at least partial unfolding of said aptamer oligonucleotide and an inhibition of the latter by said inhibitor, and
(iii) amplifying said cDNA by polymerase chain reaction.

According to the invention “target RNA” refers to said RNA molecule contained or suspected to be contained in the sample and which is intended to be amplified. Said target RNA may be of any living being origin, including animal, human, bacterial, viral or plant origin.

According to the invention the “first temperature” or, synonymously, “low temperature” refers to a temperature where the sample is stored before subjecting it to the revers transcription at step (ii). The first temperature is below the second temperature and, therefore, below the optimum temperature for the revers transcription. In an embodiment said first temperature is about ambient temperature typically prevailing in the laboratory, i.e. around 23° C. In another embodiment said first temperature is about the temperature typically prevailing in PCR devices, such as QIAcuity®, i.e. around 37° C.

According to the invention the “sample” refers to any liquid or semi-liquid sample containing or suspected of containing said target RNA, such as a biological sample, i.e. a sample of animal or human origin, including a cell maintained in culture or from a cultured cell line; a cell lysate or lysate fraction or cell extract; a solution containing one or more molecules derived from a cell, cellular material, or viral material (e.g. a polypeptide or nucleic acid); or a solution containing a naturally or non-naturally occurring nucleic acid, which is assayed as described herein. A sample may also be any body fluid or excretion or swab (for example, but not limited to, blood, urine, stool, saliva, tears, bile) that contains cells, cell components, or nucleic acids.

A “reverse transcriptase enzyme” is an enzyme used to generate complementary DNA (cDNA) from an RNA template, a process termed reverse transcription. Suitable reverse transcriptase enzymes include, without being restricted thereto, HIV-1 reverse transcriptase from human immunodeficiency virus type 1, M-MLV reverse transcriptase from the Moloney murine leukemia virus, AMV reverse transcriptase from the avian myeloblastosis virus, and telomerase reverse transcriptase. A “reverse transcriptase enzyme” may have both a reverse transcriptase activity and a DNA-dependent polymerase activity as described below. Such enzymes are known in the art, e.g. Heller et. al. (2019), Engineering of a thermostable viral polymerase using metagenome-derived diversity for highly sensitive and specific RT-PCR, Nucleic Acids Res. 47(7), pp. 3619-3630. Said “reverse transcribing” in step (ii) means incubating said reaction mixture under conditions sufficient to allow polymerization of a nucleic acid molecule complementary to a portion of said target RNA.

A “DNA polymerase enzyme” is a member of a family of enzymes that catalyze the synthesis of DNA molecules from nucleoside triphosphates. Of preference is a DNA-dependent DNA polymerase such as a Taq polymerase or other thermostable DNA polymerases. Said “amplifying” in step (iii) means incubating said reaction mixture under conditions sufficient to allow a synthesis of multiple copies of said cDNA obtained in step (ii).

As used herein, “mixture of deoxyribonucleoside triphosphates (dNTP)” refers to a RT and/or PCR conventional mixture of the deoxynucleoside triphosphates dATP, dGTP, dCTP, dTTP, dUTP, i.e. the building-blocks from which the DNA polymerase synthesizes a new DNA strand. The terms “nucleosides” and “nucleotides can be used herein interchangeably. Also included herein are modified deoxyribonucleoside triphosphates, i.e. dNTPs with a modified nucleoside or nucleotide moiety or with a detectable marker. Non-limiting examples of modified nucleotides or nucleosides are thiouridine, e.g. 2-thiouridine (s2U), 5-methylcytidine (m5C), 5-methyluridine (m5U), N1-methylpseudouridine, 2-thiouridine (s2U), N6-methyladenosine (m6A), 5-hydroxymethylcytidine, 5-hydroxymethylcytidine, 5-hydroxymethyluridine, 5-methylcytidine, 5-methoxyuridine, 5-methoxycytidine, 5-carboxymethylesteruridine, 5-formylcytidine, 5-carboxycytidine, 5-hydroxycytidine, thienoguanosine, 5-formyluridine.

A “primer” is an oligonucleotide used to hybridize to a region of a target nucleic acid, e.g. the target RNA, to facilitate the polymerization of a complementary nucleic acid. It is to be understood that a vast array of primers can be useful in the present invention, including those not specifically disclosed herein, without departing from the scope or preferred embodiments thereof. In some other embodiments, the nucleic acid primer is complementary to a portion of the target RNA.

According to the invention, said “primer configured to convert the target RNA into complementary DNA (cDNA) and/or to amplify said cDNA” refers to a primer required by the reverse transcriptase enzyme for the first step to initiate the synthesis of complementary DNA (cDNA) and/or for the second step to subsequently amplify the generated cDNA via polymerase chain reaction. Said primer can be unspecific for the first step, and/or “target specific” for the second step.

“Unspecific primer” means that said primer is not adapted to bind to a unique sequence of target RNA. For example, for the amplification of poly-A-bearing mRNA, a so-called oligo-d (T) primer could be used, i.e. several thymine bases which are complementary to the poly(A) tail at the 3′ end of mRNAs. Other well known unspecific primers are random hexamer primers.

“Target specific” means that said primer comprises a nucleotide sequence which allows a specific hybridization to a unique sequence in the target RNA and/or generated cDNA or sections thereof, thereby defining the starting point for the DNA synthesis.

In another, alternative embodiment of the invention at least one unspecific primer configured to convert the target RNA into complementary DNA (cDNA), and at least one unspecific primer configured to convert the target RNA into complementary DNA (cDNA) are used, i.e. two different at least one primers are used, an at least one unspecific (first) primer for the first step to convert the target RNA into cDNA, and an at least one specific (second) primer for the second step to amplify the cDNA.

“At least one” primer in this context or, in an embodiment, primer pair, means that one, two, three or more or even a mixture of different primers or primer pairs, respectively.

According to the invention an “aptamer oligonucleotide configured to specifically inhibit the reverse transcriptase enzyme” refers to short single-stranded RNA or DNA oligonucleotides, typically in the range of about 25-70 bases, that, at said first temperature, can bind to the reverse transcriptase. Aptamers have dissociation constants in the pico-to nanomolar range. They therefore bind to their target molecules with a strength similar to that of antibodies. This high affinity is achieved by the 3D structure of the aptamer oligonucleotide folding precisely around the binding partner (“adaptive binding”). Aptamers are produced artificially, i.e. in vitro, according to the criterion of the highest possible specific binding affinity. For this purpose, large random libraries of oligonucleotides of different base sequence are created, in the order of 10¹⁴ to 10¹⁵ different sequences per μmol. From these sequences, those that bind the desired molecule most strongly, here the reverse transcriptase enzyme, are filtered out via the “systematic evolution of ligands by exponential enrichment” (SELEX®). The aptamer candidates are mixed with immobilized enzymes and the unbound ones washed away. What remains are candidates that have a high affinity for the reverse transcriptase enzyme. These are amplified via PCR and a new cycle of binding and washing away of the weaker bound candidates is started.

A “specific inhibition” refers to a process where, at the first temperature, the aptamer oligonucleotide interacts with the reverse transcriptase enzyme, e.g. binds to the latter, in a way which reduces significantly or even abolishes its enzymatic activity.

At the first, lower temperature in step (i) the particular base-pairings between nucleotides in the aptamers are presumed to be intact, which result in secondary and tertiary RNA structures that confer the aptamer with a binding specificity for the reverse transcriptase enzyme.

“Oligonucleotides” are oligomers made up of a few nucleotides (DNA or RNA). Typically an oligonucleotide comprises about 25 to 70 nucleotides. A preferred length of an aptamer oligonucleotide is approx. 33 nucleotides, and a preferred length of an aptamer inhibitor oligonucleotide is approx. 41 nucleotides.

According to the invention, an “inhibitor of the aptamer oligonucleotide” refers to a molecule which, at the second temperature, specifically inhibits the aptamer oligonucleotide by a specific interaction, e.g. a binding, with the latter. But the inhibitor does not or does essentially not inhibit the aptamer oligonucleotide at the first temperature. “Essentially not” in this context means that, at the first temperature, the aptamer still has its binding activity by at least about 80%, preferably about 90%, further preferably about 95%, further preferably about 96%, further preferably about 97%, further preferably about 98%, further preferably about 99%, further preferably about 100%.

It is understood that the reaction mixture according to the invention may further comprise a buffer or buffer solutions conventionally used in RT PCR reactions, such as traditional PCR buffer which is compatible with the RT enzyme or RT buffer which is compatible with the DNA polymerase enzyme, e.g. Thermo Fisher® GeneAmp™ PCR Buffer #N8080006, Promega® Reverse Transcription Buffer #A356A, Thermo Fisher® M-MLV Reverse Transcriptase Buffer #18057018, etc.

In step (ii) of the method according to the invention, due to the elevated second temperature, the base pairings between nucleotides in the aptamer are destabilized or disrupted and the aptamer oligonucleotide becomes at least partially denatured or unfolded, respectively. Thereby, the aptamer oligonucleotide loses at least partially its specific three-dimensional structure, allowing the contacting of or interacting with the inhibitor of the aptamer oligonucleotide.

Step (iii) of the method according to the invention is a conventional PCR reaction where said cDNA generated in step (ii) is amplified.

It is understood that “PCR” also includes any kind of PCR, such as real-time PCR (qPCR), digital PCR (dPCR), multiplex PCR, isothermal PCR etc.

It is further understood that, in an embodiment said method according to the invention is carried out as a conventional two-step RT-PCR, while in another preferred embodiment said method according to the invention is carried out as a one-step RT-PCR.

The object underlying the invention is herewith completely solved.

In an embodiment of the method according to the invention said inhibitor of the aptamer oligonucleotide is an inhibitor oligonucleotide capable of at least partially hybridizing to said—at the second temperature—at least partially unfolded aptamer oligonucleotide.

This measure has the advantage that such an inhibitor molecule is provided which exerts its inhibitory activity on the aptamer oligonucleotide at the second, elevated temperature by annealing to the latter. The inhibitor oligonucleotide comprises a nucleotide sequence which is at least partially or essentially complementary, preferably fully complementary to the sequence of the aptamer oligonucleotide. The hybridization of the inhibitor oligonucleotide to the aptamer oligonucleotide prevents the folding of aptamer in its three-dimensional “active” configuration and, thereby, reliably abolishes the binding affinity of the aptamer to the reverse transcriptase enzyme.

In another embodiment of the invention the aptamer oligonucleotide is an aptamer RNA oligonucleotide and/or in another embodiment said inhibitor oligonucleotide is an inhibitor DNA oligonucleotide.

The aptamer can be made of DNA, RNA, LNA or even of mixed bases. The use of an aptamer RNA oligonucleotide has the advantage of obtaining useful secondary and tertiary structures. The inhibitor oligonucleotide can also be made of DNA, RNA, LNA or even of mixed bases. The use of an inhibitor DNA oligonucleotide has the advantage that the molecule can be generated in a fast and cheap manner and the likelihood of forming secondary structures is reduced.

In still another embodiment of the invention said inhibitor oligonucleotide comprises a nucleotide sequence which is at least 60%, preferably at least 70%, preferably 80%, preferably 90%, and preferably 100% complementary with the nucleotide sequence of said aptamer oligonucleotide.

By this measure such an inhibitor oligonucleotide is provided which ensures an effective inhibition of the aptamer oligonucleotide at the second, elevated temperature.

A nucleotide sequence which is “at least 60% complementary” refers to a sequence having, over its entire length, at least about 88%, or more, in particular at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 8% 3, 84%, 85%, 86%, 87%, 88%, 89%, 90%, 91%, 92%, 93%, 94%, 95%, 96%, 97%, 98%, 99% or 100% sequence complementarity with the entire length of a reference sequence, e.g. the aptamer sequence.

A nucleotide sequence of at least 60% complementarity correspond to a nucleotide sequence having at least 60% homology to a perfectly, i.e. 100% complementary nucleotide sequence.

Methods for comparing the complementarity/homology/identity of two or more sequences are known in the art. For example, the “needle” program, which uses the Needleman-Wunsch global alignment algorithm (Needleman and Wunsch, 1970 J. Mol. Biol. 48:443-453) to find the optimum alignment (including gaps) of two sequences when considering their entire length may be used. The needle program is for example available on 30 the World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the disclosure, is calculated using the EMBOSS: needle (global) program with a “Gap Open” parameter equal to 10.0, a “Gap Extend” parameter equal 35 to 0.5, and a Blosum62 matrix.

In an embodiment of the method according to the invention said aptamer oligonucleotide comprises the following nucleotide sequence:

(SEQ ID NO: 1)
5′-UUACCACGCGCUCUUAACUGCUAGCGCCAUGGC-3′.

By this measure such an aptamer RNA sequence is provided which effectively inhibits the reverse transcriptase enzyme at the first, lower temperature, especially the MMLV reverse transcriptase.

In a further embodiment of the method according to the invention said inhibitor oligonucleotide comprises the following nucleotide sequence:

(SEQ ID NO: 2)
5′-AGTTTTGGCCATGGCGCTAGCAGTTAAGAGCGCGTGGTAAG-3′.

By this measure such an inhibitor nucleotide sequence is provided which effectively inhibits the aptamer at the second, elevated temperature.

In another embodiment of the invention the nucleotide sequence of said aptamer oligonucleotide and/or said inhibitor oligonucleotide comprises at its 3′ terminus a phosphate modification (PO4).

This measure has the advantage that the 3′ PO4 prevents the aptamer and aptamer inhibitor oligonucleotide from serving as primers themselves for the RT or PCR reactions. The 3′ PO4 blocks DNA polymerases from adding on nucleotides.

In still another embodiment of the method according to the invention said DNA polymerase enzyme is a thermostable DNA polymerase, preferably a DNA-dependent DNA polymerase.

This measure uses in an advantageous manner such a DNA polymerase which is optimized for a use in PCR. E.g. Taq polymerase, a prototype of a thermostable DNA-dependent DNA polymerase, has an optimum working temperature at about 75° C.-80° C. but also works at 50° C., and therefore does not degenerate and can be used at the thermocycling reactions in a PCR or RT-PCR.

In another embodiment of the invention said second, elevated temperature is higher than or equal to approx. 45° C., preferably higher than or equal to approx. 47° C., further preferably equal to approx. 50° C.

This measure uses in an advantageous manner such a temperature which results in an at least partial unfolding of the aptamer oligonucleotide, thereby allowing the inhibitor oligonucleotide to hybridize to the aptamer oligonucleotide and to effectively block its binding capacity. At the same time, the elevated temperature ensures a specific annealing of the primers. It also breaks down secondary structures of the target RNA. Thereby, the second, elevated temperature ensures an effective and specific reverse transcription and PCR amplification. In this context “higher than” means that there is no upper temperature limit. There are thermostable RT enzymes active at temperatures ranging from 60° C. to 70° C. for which similar aptamer/inhibitor oligomers could be developed. Therefore, according to the invention for the second, elevated temperature values are also included of, e.g., 47° C., 48° C., 49° C., 50° C., 51° C., 52° C., 53° C., 54° C., 55° C., 56° C., 57° C., 58° C., 59° C., 60° C., 65° C., 70° C., 75° C., . . . , etc.

In yet another embodiment of the method according to the invention said first, lower temperature is less than or equal to approx. 47° C., preferably less than or equal to approx. 45° C., further preferably less than or equal to approx. 43° C., more preferably less than or equal to approx. 37° C., and highly preferably less than or equal approx. 23° C.

This measure ensures an effective blocking of the reverse transcriptase enzyme by the aptamer oligonucleotide as the latter takes its three-dimensional structure, thereby having maximum binding affinity to the reverse transcriptase enzyme. It is to be understood that the lower the first temperature is the more preferably it is. “Less than approx. 47° C.” in this context means approx. 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 10° C., 5° C., . . . , etc.

In an embodiment of the method according to the invention the reverse transcriptase enzyme is Moloney murine leukemia virus reverse transcriptase (MMLV-RT).

By this measure a well-established and reliable reverse transcriptase enzyme is employed. MMLV-RT is a recombinant DNA polymerase that synthesizes a DNA complementary strand from single-stranded RNA, DNA, or an RNA: DNA hybrid. Compared to Avian myeloblastosis virus reverse transcriptase (AMV-RT), MMLV-RT has only minor DNA endonuclease and RNase H activity. It is thermostable and has optimal activity at 37° C. or even at higher temperatures, e.g. 50° C. MMLV-RT can be used to synthesize first-strand cDNA up to 7 kb.

Another subject-matter of the invention relates to an oligonucleotide molecule, i.e. the inhibitor oligonucleotide characterized above, comprising the following nucleotide sequence:

(SEQ ID NO: 2)
5′-AGTTTTGGCCATGGCGCTAGCAGTTAAGAGCGCGTGGTAAG-3′.

The features, characteristics, advantages and embodiments disclosed for the method according to the invention apply to the oligonucleotide correspondingly.

Another subject-matter of the invention relates to a kit for amplifying a target RNA, comprising:
a reverse transcriptase enzyme;
a DNA polymerase enzyme;
a mixture of deoxyribonucleoside triphosphates (dNTPs);
at least one primer configured to convert the target RNA into complementary DNA (cDNA) and/or to amplify said cDNA;
an aptamer oligonucleotide configured to specifically inhibit the reverse transcriptase enzyme, and
an inhibitor of the aptamer oligonucleotide.

A kit is a combination of individual elements useful for carrying out the method of the invention, wherein the elements are optimized for use together in the method. The kit also contains a manual for performing the method according to the invention. Such kits unify all essential elements required to work the method according to the invention, thus minimizing the risk of errors. Therefore, such kits also allow semiskilled laboratory staff to perform the method according to the invention.

The features, characteristics, advantages and embodiments disclosed for the method according to the invention apply to the kit correspondingly.

Another subject-matter of the invention relates to a use of an oligonucleotide for inhibiting an aptamer oligonucleotide configured to specifically inhibit a reverse transcriptase enzyme.

The features, characteristics, advantages and embodiments disclosed for the method according to the invention apply to the use correspondingly.

In an embodiment of the use according to the invention said inhibition of said aptamer oligonucleotide occurs in a temperature-dependent manner.

“Temperature-dependent” refers to an inhibition activity of said aptamer oligonucleotide, which may or may not be exerted by the oligonucleotide, depending on the prevailing temperature conditions. E.g. at the first lower temperature as defined above, the inhibition activity of said aptamer oligonucleotide may prevail or is prevailing, while at the second, elevated temperature as defined above the inhibition activity of the aptamer may be or is absent.

It is to be understood that the before-mentioned features and those to be mentioned in the following cannot only be used in the combination indicated in the respective case, but also in other combinations or in an isolated manner without departing from the scope of the invention.

### EMBODIMENTS

The invention is now further explained by means of embodiments resulting in additional features, characteristics and advantages of the invention. The embodiments are of pure illustrative nature and do not limit the scope or range of the invention. The features mentioned in the specific embodiments are features of the invention and may be seen as general features which are not applicable in the specific embodiment but also in an isolated manner in the context of any embodiment of the invention.

### Embodiment 1

To develop a universal warm start RT-PCR chemistry, the inventor pursued an approach that would rely on an oligonucleotide aptamer to bind to and inhibit RT enzymes at lower temperatures prior to RT-PCR cycling, while allowing full RT activity during RT-PCR cycling. Two publications (Chen and Gold, 1994, I.c.; Rutschke et al., 2015, I.c.) describe an RNA aptamer generated via SELEX that specifically binds the Moloney murine leukemia virus reverse transcriptase (MMLV-RT). In this application, that RNA aptamer will be referred to as MMLV-RT RNA aptamer (Table 1).

TABLE 1
Sequences of oligonucleotides described in this invention. The
sequence of the RNA aptamer (MMLV-RT RNA aptamer) published in Chen
and Gold, 1994, l.c., and Rutschke et al., 2015, l.c., with a 3′
phosphate modification, that specifically binds MMLV-RT enzyme, is
provided. The sequence of the inhibitor DNA oligonucleotide (MMLV-
RT RNA aptamer Inhibitor) that dramatically reduces the extent to
which the MMLV-RT RNA aptamer inhibits MMLV-RT activity at 50° C.
is also provided.

| Oligo Name | Oligo Sequence (5′ to 3′) |
|---|---|
| MMLV-RT RNA aptamer | 5′-UUACCACGCGCUCUUAACUGCUAGCGCCAUGGC-PO4-3′ |
| | (SEQ ID NO: 1) |
| MMLV-RT RNA aptamer | 5′- |
| inhibitor | AGTTTTGGCCATGGCGCTAGCAGTTAAGAGCGCGTGGTAAG- |
| | PO4-3′ (SEQ ID NO: 2) |

When added to one-step RT-qPCR reactions, it has been shown that the MMLV-RT RNA aptamer can provide a warm start RT function to specific one-step RT-qPCR applications (Rutschke et al., 2015, I.c.).

To evaluate the MMLV-RT RNA aptamer for the given needs, the inventor gave particular consideration to QIAcuity one-step RT-dPCR workflows (Qiagen®, Hilden, Germany). These workflows are especially thermally challenging, given the relatively warm temperatures of about 37° C. found inside QIAcuity Nanoplate trays, where dPCR plates can wait for hours before being cycled. The inventor found that any warm-start RT solution that would work with QIAcuity one-step RT-dPCR workflows would also address the warm start RT needs of standard RT-PCR applications, including but not limited to one-step RT-qPCR.

When added to multiplex one-step RT-dPCR reactions using hydrolysis probes to detect PCR amplification and containing MMLV-RT enzyme, the MMLV-RT RNA aptamer was able to suppress MMLV-RT activity in a temperature, concentration, and assay dependent manner, relative to control reactions containing MMLV-RT but no aptamer (Table 2A,B). However, these RNA aptamers also inhibited RT activity at optimal and desired temperatures (50° C.) when compared to control reactions without aptamer (Table 2A).

To address the unwanted inhibition of the MMLV-RT activity by the MMLV-RT RNA aptamer at 50° C., the inventor considered the temperature dependent manner in which RNA-aptamers, such as MMLV-RT RNA aptamer, are thought to specifically bind with their substrates. At lower temperatures particular base-pairings between nucleotides in the aptamers are presumed to be intact, which result in secondary and tertiary RNA structures that confer the aptamer with a binding specificity for particular target substrates. As temperatures rise, these base pairings are destabilized or disrupted, such that the aptamer partially or entirely unfolds and loses its ability to bind its target substrate.

Assuming that aptamers primarily exist in either folded/functional and unfolded/nonfunctional states, the inventor proposed that introducing an oligonucleotide (MMLV-RT RNA aptamer inhibitor, Table 1) containing a reverse-complement sequence to the MMLV-RT RNA aptamer could be used to capture unfolded or partially unfolded MMLV-RT RNA aptamers at higher temperatures (e.g. 50° C.). Once annealed to the oligonucleotide, the inventor supposed that the MMLV-RT RNA aptamer would lose its ability to refold and inhibit the MMLV-RT. At lower temperatures, the inventor anticipated that the stable secondary and tertiary structures of the MMLV-RT RNA aptamer would largely prevent the MMLV-RT RNA aptamer inhibitor from binding. Thus, at lower temperatures, the folded MMLV-RT RNA aptamer could sufficiently inhibit unwanted RT activity at lower temperatures, even in the presence of the MMLV-RT RNA aptamer inhibitor.

In multiplex QIAcuity one-step RT-dPCR reactions using hydrolysis probes to detect PCR amplification and containing MMLV-RT and the MMLV-RT RNA aptamer at concentrations between 0.05 and 0.25 μM, addition of 0.5 μM MMLV-RT RNA aptamer inhibitor was able to entirely eliminate or dramatically reduce aptamer-dependent inhibition of MMLV-RT activity at 50° C. (Table 2C). Strikingly, restoration of full RT activity at 50° C. did not come at the expense of “warm start” RT functionality, as 78% to 100% of RT activity could still be suppressed in the NO RT Step reaction when the Inhibitor was present (Table 2D).

TABLE 2
In QIAcuity one-step RT-dPCR reactions using hydrolysis
probes, MMLV-RT RNA aptamer inhibits unwanted MMLV-RT
activity at lower temperatures as well as desired RT activity
at 50° C. Introducing an inhibitor oligo to these
reactions ensures optimal RT activity at 50° C. while
leaving inhibition of RT at lower temperatures intact.
A

| | | 50° C. RT Step | | | |
|---|---|---|---|---|---|
| | | MMLV-RT RNA Aptamer | | | |
| | | 0 μM | 0.05 μM | 0.1 μM | 0.25 μM |
| Assay | GAPDH | 100% | 66% | 41% | 19% |
| | N1 | 100% | 96% | 88% | 69% |
| | QNIC | 100% | 86% | 60% | 34% |

B

| | | NO RT Step | | | |
|---|---|---|---|---|---|
| | | MMLV-RT RNA Aptamer | | | |
| | | 0 μM | 0.05 μM | 0.1 μM | 0.25 μM |
| Assay | GAPDH | 88% | 0% | 0% | 0% |
| | N1 | 56% | 9% | 7% | 5% |
| | QNIC | 76% | 2% | 1% | 1% |

C

| | | 50° C. RT Step | | | |
|---|---|---|---|---|---|
| | | MMLV-RT RNA Aptamer | | | |
| | | 0 μM | 0.05 μM | 0.1 μM | 0.25 μM |
| | | MMLV-RT RNA Aptamer Inhibitor | | | |
| | | 0.5 μM | 0.5 μM | 0.5 μM | 0.5 μM |
| Assay | GAPDH | 99% | 98% | 92% | 84% |
| | N1 | 96% | 96% | 96% | 94% |
| | QNIC | 103% | 107% | 104% | 105% |

D

| | | NO RT Step | | | |
|---|---|---|---|---|---|
| | | MMLV-RT RNA Aptamer | | | |
| | | 0 μM | 0.05 μM | 0.1 μM | 0.25 μM |
| | | MMLV-RT RNA Aptamer Inhibitor | | | |
| | | 0.5 μM | 0.5 μM | 0.5 μM | 0.5 μM |
| Assay | GAPDH | 87% | 1% | 0% | 0% |
| | N1 | 55% | 22% | 13% | 7% |
| | QNIC | 77% | 10% | 3% | 1% |

Relative levels of quantification for GAPDH (Jurkat cell RNA), N1 (synthetic SARS-COV-2 genomic RNA), and QNIC (synthetic RNA) in multiplex QIAcuity RT-dPCR are shown for reaction mixes cycled in two different manners. To capture full RT activity at 50° C., reaction mixes were pipetted into Nanoplates that were directly inserted into a QIAcuity instrument and immediately cycled with a standard RT-dPCR cycling program that contained a 40 minute 50° C. RT step (Table 2A,C). To capture the levels of reverse transcription that occur while Nanoplates wait for hours to be cycled in the QIAcuity instrument, reaction mixes were incubated in Nanoplates in a QIAcuity instrument for 2 hours prior to dPCR cycling that lacked a 50° C. RT step (NO RT Step) (B,D). RNA target quantification was normalized to control reactions containing neither MMLV-RT RNA aptamer nor MMLV-RT RNA aptamer inhibitor which were directly cycled with a standard RT-dPCR cycling program (Table 2A, 0 pM).

In NO RT step reactions that did not contain the MMLV-RT RNA aptamer (Table 2B, 0 μM), 56 to 88% of RT activity could be detected, compared to control. This reflects the high levels of reverse transcription that occur when Nanoplates must wait for hours before being cycled. When the MM LV-RT RNA aptamer was present in these reaction mixes at concentrations of 0.05 μM to 0.25 μM, 90% to 100% of RT activity could be blocked. The extent to which the MMLV-RT aptamer inhibits RT activity at lower temperatures is aptamer concentration and assay dependent (Table 2B). However, the presence of the MMLV-RT RNA aptamer also inhibits desired RT activity at 50° C. Depending on the aptamer concentration and assay, the MMLV-RT RNA aptamer inhibits 4 to 81% of RT activity at 50° C. (Table 2A).

Remarkably, when an inhibitor oligonucleotide (MMLV-RT RNA aptamer inhibitor) is introduced to these reactions at a concentration of 0.5 μM, full RT efficiency at 50° C. could be recovered (C) while maintaining the ability of the MMLV-RT RNA aptamer to potently inhibit RT at lower temperatures (D).

### Embodiment 2

The ability of the MMLV-RT RNA aptamer inhibitor to reduce or eliminate the inhibition of RT activity at 50° C. in the presence of the MMLV-RT RNA aptamer also works in one-step RT-qPCR applications. Multiplex one-step RT-qPCR reactions were performed with a thermal cycler programed to have a defined gradient of temperatures, ranging from 33° C. to 55° C., across the thermal cycler heating block during the 40 min RT step of the RT-qPCR cycling program. Thus, wells containing the same reaction mix could be incubated at different temperatures during the RT step, allowing one to determine relative RT activity over a range of temperatures. Compared to control reactions (Table 3A,B) addition of 1 μM MMLV-RT RNA aptamer inhibitor to reaction mixes containing MMLV-RT and 0.1 μM MMLV-RT RNA aptamer was sufficient to entirely eliminate or dramatically reduce aptamer-dependent inhibition of MMLV-RT activity at 50° C. (Table 3C,D). Strikingly, restoration of full RT activity at 50° C. did not come at the expense of “warm start” RT functionality, as RT activity at lower could still be suppressed at lower temperatures when aptamer inhibitor was present (Table 3C,D).

TABLE 3
MMLV-RT RNA aptamer inhibitor can reactivity
at 50° C. in the presence of the MMLV-RT
RNA aptamer in one-step RT-qPCR applications.

| | A | B | C | D |
|---|---|---|---|---|
| MMLV-RT RNA aptamer | NONE | NONE | 0.1 μM | 0.1 μM |
| concentration | | | | |
| MMLV-RT RNA aptamer | NONE | 1 μM | NONE | 1 μM |
| inhibitor concentration | | | | |
| GAPDH FAM | | | | |
| 55° C. | 66% | 71% | 51% | 57% |
| 54.5° C. | 67% | 75% | 66% | 67% |
| 53.9° C. | 69% | 82% | 70% | 78% |
| 52.9° C. | 90% | 97% | 80% | 95% |
| 51.4° C. | 97% | 97% | 79% | 94% |
| 49.7° C. | 100% | 96% | 72% | 94% |
| 47.6° C. | 100% | 96% | 42% | 91% |
| 45.4° C. | 96% | 90% | 18% | 87% |
| 42.6° C. | 88% | 89% | 8% | 73% |
| 40.0° C. | 88% | 82% | 4% | 39% |
| 38.5° C. | 85% | 81% | 2% | 18% |
| 36.8° C. | 82% | 80% | 1% | 7% |
| 35.3° C. | 93% | 75% | 1% | 4% |
| 34.2° C. | 81% | 80% | 1% | 2% |
| 33.4° C. | 76% | 78% | 1% | 2% |
| 33.0° C. | 75% | 77% | 1% | 2% |
| N1 Cy5 | | | | |
| 55° C. | 60% | 80% | 80% | 91% |
| 54.5° C. | 59% | 78% | 96% | 84% |
| 53.9° C. | 78% | 90% | 94% | 93% |
| 52.9° C. | 89% | 93% | 98% | 93% |
| 51.4° C. | 93% | 98% | 94% | 97% |
| 49.7° C. | 100% | 99% | 94% | 113% |
| 47.6° C. | 104% | 97% | 77% | 103% |
| 45.4° C. | 89% | 86% | 57% | 86% |
| 42.6° C. | 59% | 66% | 30% | 66% |
| 40.0° C. | 52% | 52% | 18% | 49% |
| 38.5° C. | 44% | 45% | 11% | 36% |
| 36.8° C. | 34% | 34% | 7% | 19% |
| 35.3° C. | 37% | 29% | 7% | 13% |
| 34.2° C. | 30% | 29% | 6% | 9% |
| 33.4° C. | 29% | 27% | 5% | 7% |
| 33.0° C. | 28% | 27% | 5% | 7% |
| QN IC VIC | | | | |
| 55° C. | 33% | 47% | 55% | 53% |
| 54.5° C. | 38% | 55% | 62% | 70% |
| 53.9° C. | 50% | 66% | 94% | 95% |
| 52.9° C. | 68% | 97% | 110% | 106% |
| 51.4° C. | 99% | 101% | 109% | 122% |
| 49.7° C. | 100% | 110% | 91% | 109% |
| 47.6° C. | 100% | 103% | 56% | 113% |
| 45.4° C. | 106% | 104% | 27% | 97% |
| 42.6° C. | 91% | 87% | 12% | 70% |
| 40.0° C. | 74% | 75% | 6% | 50% |
| 38.5° C. | 65% | 60% | 4% | 24% |
| 36.8° C. | 57% | 57% | 3% | 13% |
| 35.3° C. | 53% | 54% | 3% | 7% |
| 34.2° C. | 57% | 54% | 3% | 5% |
| 33.4° C. | 56% | 51% | 2% | 4% |
| 33.0° C. | 53% | 50% | 2% | 5% |

Relative levels of quantification for GAPDH (Jurkat cell RNA), N1 (synthetic SARS-COV-2 genomic RNA), and QNIC (synthetic RNA) in multiplex one-step RT-qPCR reactions are shown for reaction mixes cycled with a thermal cycler programed to have a defined gradient of temperatures, ranging from 33° C. to 55° C., across the thermal cycler heating block during the 40 min RT step of the RT-qPCR cycling program. RNA target quantification was normalized to the Cq values for control reactions containing neither MMLV-RT RNA aptamer nor MM LV-RT RNA aptamer inhibitor which were incubated at 49.7° C. during the RT (Table 3A, bold).

In control reactions that did not contain the MMLV-RT RNA Aptamer (Table 3A, B), 28 to 75% of RT activity could be detected in reactions incubated at 33° C. This reflects the high levels of reverse transcription that occurs at lower temperatures. When the MMLV-RT RNA aptamer was present in these reaction mixes at concentrations of 0.1 μM, 95% to 99% of RT activity could be blocked. The extent to which the MMLV-RT aptamer inhibits RT activity at lower temperatures is aptamer-concentration and assay dependent (Table 3C). However, the presence of the MMLV-RT RNA aptamer also inhibits desired RT activity at ˜50° C. Depending on the aptamer concentration and assay, the MMLV-RT RNA aptamer inhibits 6 to 28% of RT activity at 50° C. in these reactions (Table 3C).

Remarkably, when an inhibitor oligonucleotide (MMLV-RT RNA aptamer inhibitor) is introduced to these reactions at a concentration of 1 μM, full RT efficiency at 50° C. could be recovered (Table 3D) while maintaining the ability of the MMLV-RT RNA aptamer to potently inhibit RT at lower temperatures (Table 3D). Addition of the inhibitor oligonucleotide alone had no negative impact on one-step RT-qPCR performance (Table 3B), and was observed to confer the MMLV-RT enzyme with the ability to more efficiently reverse transcribe at higher temperatures (>50° C.) compared to control.

### Embodiment 3

The ability of the MMLV-RT RNA aptamer inhibitor to reduce or eliminate the inhibition of RT activity at 50° C. in the presence of the MMLV-RT RNA aptamer is also compatible with one-step RT-dPCR applications that use intercalating dyes to detect PCR amplification (such as SYBR Green and EvaGreen). This is not obvious, as the MMLV-RT RNA aptamer inhibitor/MMLV-RT RNA aptamer complex could strongly bind intercalating dyes. This could increase the baseline (i.e background) fluorescence in the dPCR such that fluorescent signal from amplified PCR products could not be distinguished from baseline fluorescence. Furthermore, it is possible that the addition of intercalating dyes could interfere with the interactions of the MMLV-RT RNA aptamer inhibitor/MMLV-RT RNA aptamer complex, such that the benefits of the MMLV-RT RNA aptamer inhibitor are diminished or lost.

To address these questions, QIAcuity one-step RT-dPCR reactions containing EvaGreen intercalating dye, MMLV-RT, and 0.05 μM MMLV-RT RNA aptamer were assembled and used to quantify the abundance of the human mRNA PPIA. Consistent with data from RT-dPCR using hydrolysis probes, addition of 0.1 μM MMLV-RT RNA aptamer inhibitor was able to eliminate aptamer-dependent inhibition of MMLV-RT activity at 50° C. (Table 4, upper part). Restoration of full RT activity at 50° C. did not come at the expense of “warm start” RT functionality, as 94% of RT activity could still be suppressed in the NO RT Step reaction when the Inhibitor was present (Table 4, lower part).

TABLE 4
Elimination of aptamer-dependent inhibition of MMLV-RT
activity at 50° C. by addition of inhibitor; no
restoration of full RT activity in NO RT Step reaction.

| | Human |
|---|---|
| | PPIA |
| | Rel. Quant. |
| 50° C. RT Step | |
| No MMLV-RT RNA Aptamer | |
| 0.0 μM MMLV-RT RNA Aptamer inhibitor | 100% |
| 0.1 μM MMLV-RT RNA Aptamer inhibitor | 103% |
| 0.05 μM MMLV-RT RNA Aptamer | |
| 0.0 μM MMLV-RT RNA Aptamer inhibitor | 80% |
| 0.1 μM MMLV-RT RNA Aptamer inhibitor | 100% |
| NO RT Step | |
| No MMLV-RT RNA Aptamer | |
| 0.0 μM MMLV-RT RNA Aptamer inhibitor | 40% |
| 0.1 μM MMLV-RT RNA Aptamer inhibitor | 50% |
| 0.05 μM MMLV-RT RNA Aptamer | |
| 0.0 μM MMLV-RT RNA Aptamer inhibitor | 6% |
| 0.1 μM MMLV-RT RNA Aptamer inhibitor | 6% |

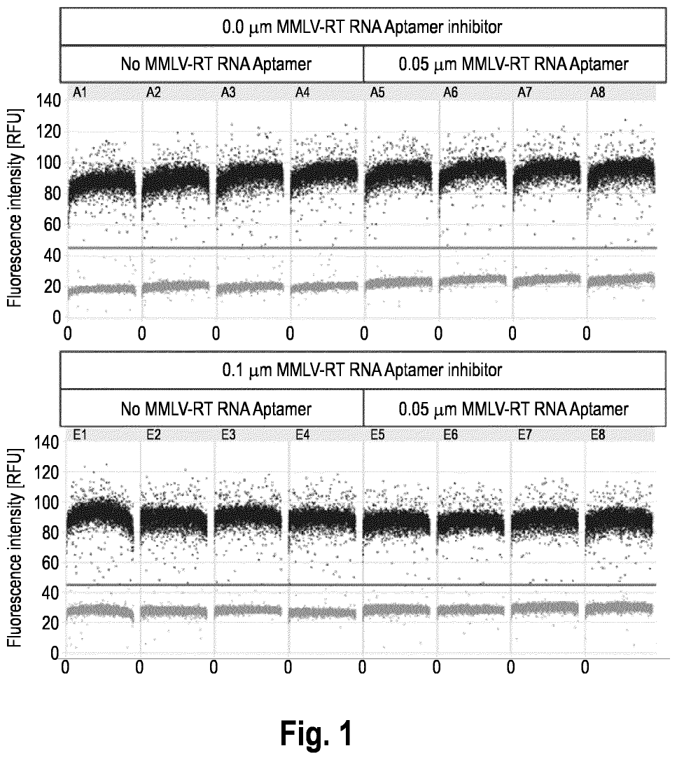

Most importantly, baseline fluorescence in reactions containing 0.05 μM MMLV-RT RNA and 0.1 μM MMLV-RT RNA aptamer inhibitor was similar to baseline fluorescence of reactions that did not contain 0.05 μM MMLV-RT RNA or 0.1 μM MMLV-RT RNA aptamer inhibitor (FIG. 1).

1-15. (canceled)

16. A method for amplifying a target RNA in a sample comprising the following steps:
(i) providing a reaction mixture at a first temperature, said reaction mixture comprising:
the sample;
a reverse transcriptase enzyme;
a DNA polymerase enzyme;
a mixture of deoxyribonucleoside triphosphates (dNTPs);
at least one primer configured to convert the target RNA into complementary DNA (cDNA) and/or to amplify said cDNA;
an aptamer oligonucleotide configured to specifically inhibit the reverse transcriptase enzyme, and
an inhibitor of the aptamer oligonucleotide,
(ii) reverse transcribing said target RNA into cDNA by incubating said reaction mixture at a second temperature higher than said first temperature resulting in an at least partial unfolding of said aptamer oligonucleotide and an inhibition of the latter by said inhibitor, and
(iii) amplifying said cDNA by polymerase chain reaction.

17. The method of claim 16, wherein said inhibitor of the aptamer oligonucleotide is an inhibitor oligonucleotide capable of at least partially hybridizing to said at least partially unfolded aptamer oligonucleotide at said second temperature.

18. The method of claim 16, wherein said aptamer oligonucleotide is an aptamer RNA oligonucleotide.

19. The method of claim 17, wherein said inhibitor oligonucleotide is an inhibitor DNA oligonucleotide.

20. The method of claim 17, wherein said inhibitor oligonucleotide comprises a nucleotide sequence which is at least 60% complementary with the nucleotide sequence of said aptamer oligonucleotide.

21. The method of claim 17, wherein said inhibitor oligonucleotide comprises a nucleotide sequence which is at least 70% complementary with the nucleotide sequence of said aptamer oligonucleotide.

22. The method of claim 17, wherein said inhibitor oligonucleotide comprises a nucleotide sequence which is at least 80% complementary with the nucleotide sequence of said aptamer oligonucleotide.

23. The method of claim 17, wherein said inhibitor oligonucleotide comprises a nucleotide sequence which is at least 90% complementary with the nucleotide sequence of said aptamer oligonucleotide.

24. The method of claim 17, wherein said inhibitor oligonucleotide comprises a nucleotide sequence which is 100% complementary with the nucleotide sequence of said aptamer oligonucleotide.

25. The method of claim 16, wherein said aptamer oligonucleotide comprises the following nucleotide sequence:
(SEQ ID NO: 1)
5′-UUACCACGCGCUCUUAACUGCUAGCGCCAUGGC-3′.

26. The method of claim 17, wherein said inhibitor oligonucleotide comprises the following nucleotide sequence:
(SEQ ID NO: 2)
5′-AGTTTTGGCCATGGCGCTAGCAGTTAAGAGCGCGTGGTAAG-3′.

27. The method of claim 26, wherein said aptamer oligonucleotide comprises the following nucleotide sequence:
(SEQ ID NO: 1)
5′-UUACCACGCGCUCUUAACUGCUAGCGCCAUGGC-3′.

28. The method of claim 27, wherein the nucleotide sequence of said aptamer oligonucleotide and/or said inhibitor oligonucleotide comprises at its 3′ terminus a phosphate modification (PO4).

29. The method of claim 16, wherein said DNA polymerase enzyme is a thermostable DNA polymerase.

30. The method of claim 29, wherein the thermostable DNA polymerase is a DNA dependent DNA polymerase.

31. The method of claim 16, wherein said second temperature is higher than or equal to approx. 45° C.

32. The method of claim 16, wherein said second temperature is higher than or equal to approx. 47° C.

33. The method of claim 16, wherein said second temperature is higher than or equal to approx. 50° C.

34. The method of claim 16, wherein said first temperature is less than or equal to approx. 47° C.

35. The method of claim 16, wherein said first temperature is less than or equal to approx. 45° C.

36. The method of claim 16, wherein said first temperature is less than or equal to approx. 43° C.

37. The method of claim 16, wherein said first temperature is less than or equal to approx. 37° C.

38. The method of claim 16, wherein the reverse transcriptase enzyme is Moloney Murine Leukemia Virus Reverse Transcriptase (MMLV-RT).

39. An oligonucleotide comprising the following nucleotide sequence:
(SEQ ID NO: 2)
5′-AGTTTTGGCCATGGCGCTAGCAGTTAAGAGCGCGTGGTAAG-3′.

40. A kit for amplifying a target RNA, comprising:
a reverse transcriptase enzyme;
a DNA polymerase enzyme;
a mixture of deoxyribonucleoside triphosphates (dNTPs);
at least one primer configured to convert the target RNA into complementary DNA (cDNA) and/or to amplify said cDNA;
an aptamer oligonucleotide configured to specifically inhibit the reverse transcriptase enzyme, and
an inhibitor of the aptamer oligonucleotide.
